(19) United States
(12) Patent Application Publication
(10) Pub. No.: US 2026/0011416 A1
(43) Pub. Date: Jan. 8, 2026

(54) SYSTEMS, DEVICES, AND METHODS FOR INTEGRATION OF ANALYTE MONITORING DATA AND ELECTRONIC MEDICAL RECORD SYSTEMS

(71) Applicant: ABBOTT DIABETES CARE INC., Alameda, CA (US)

(72) Inventors: Claire B. Weiler, San Francisco, CA (US); Patrick Wells, Bend, OR (US)

(21) Appl. No.: 19/133,933

(22) PCT Filed: Dec. 8, 2023
(86) PCT No.: PCT/US2023/083187

§ 371 Date: May 29, 2025

Related U.S. Application Data
(60) Provisional application No. 63/431,298, filed on Dec. 8, 2022.

(51) Int. Cl. G16H 10/60; G16H 80/00

(52) U.S. Cl. CPC G16H 10/60; G16H 80/00

(57) ABSTRACT

Systems and methods for integrating analyte monitoring system data and electronic medical record system data are described. The methods include associating a patient's analyte data identified with an external patient identifier associated with at least one healthcare practice. The patient's analyte data identified with the external patient identifier may be transferred from a first cloud server to a second cloud server. The analyte data may be stored in a folder associated with the at least one healthcare practice in the second cloud server. The analyte data may also be merged with electronic medical records of the patient in the second cloud server. The merged data may then be transferred to a database for further analysis.

### FIELD

The subject matter described herein relates generally to systems, devices, and methods relating to the integration of analyte monitoring data and electronic medical record systems.

### BACKGROUND

The detection and/or monitoring of analyte levels, such as glucose, ketones, lactate, oxygen, hemoglobin A1C, or the like, can be vitally important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor and can be applied by the individual with a sensor applicator. The application process includes inserting at least a portion of a sensor that senses a user's analyte level in a bodily fluid located in a layer of the human body, using an applicator or insertion mechanism, such that the sensor comes into contact with a bodily fluid. The sensor control device may also be configured to transmit analyte data to another device, from which the individual, her health care provider (“HCP”), or a caregiver can review the data and make therapy decisions.

In certain environments, such as hospital settings, other relevant patient information, such as other physiological measurements, diagnoses, and/or treatment plans, to name only a few, are collected about the patient. Such information is often stored in an electronic medical record system that is separate and independent from the in vivo analyte monitoring system that collects the patient's analyte data. Correlating information and data between these separate systems can provide insights not only at the individual level, but also with a variety of population and epidemiological studies. At the same time, the patient's privacy should also be adequately protected.

Thus, needs exist for improved methods for integrating analyte monitoring systems and electronic medical record systems, as well as methods and devices relating thereto, that are robust and secure.

### SUMMARY

Provided herein are example embodiments of improvements in systems, methods, and devices for integrating analyte monitoring systems and electronic medical record systems. The present invention provides methods as defined in the appended claims 1 and 16, and a system as defined in claim 15. According to many embodiments, methods of integrating analyte monitoring system data and electronic medical records system data includes the steps of: receiving, by a first trusted computer system, data indicative of an analyte level of a patient from an analyte monitoring system; associating, by the first trusted computer system, the data indicative of the analyte level of the patient with an external patient identifier associated with at least one practice to create an external patient identifier analyte data set; transferring, from the first trusted computer system to a second trusted computer system, the external patient identifier analyte data set; storing, by the second trusted computer system, the external patient identifier analyte data set in a folder associated with the at least one practice; transferring, from the second trusted computer system to a site server associated with the at least one practice, the external patient identifier analyte data set; merging, by the site server, the external patient identifier analyte data set with an electronic medical record associated with the patient to create an external patient identifier medical data set; and transferring, by the site server, the external patient identifier medical data set to a database.

According to an aspect of some embodiments, the external patient identifier analyte data set does not include identity information of the user.

According to an aspect of some embodiments, at least a portion of the data related to the analyte levels of the user associated with the external patient identifier comprises historical glucose results.

According to an aspect of some embodiments, the at least a portion of the data related to the analyte levels of the user associated with the external patient identifier comprises glucometrics. According to an aspect of some embodiments, the glucometrics comprises at least one of average glucose levels, time in range, and standard deviation.

According to an aspect of some embodiments, the at least a portion of the data related to the analyte levels of the user associated with the external patient identifier comprises an ambulatory glucose profile.

According to an aspect of some embodiments, the external patient identifier is assigned by the at least one practice.

According to an aspect of some embodiments, the data indicative of the analyte level is associated with a plurality of practices. According to an aspect of some embodiments, the second trusted computer system transfers the external patient identifier analyte data set to a site server associated with each of the plurality of practices.

According to an aspect of some embodiments, the method further includes the step of inviting a user to share data with the at least one practice before the step of associating, by the first trusted computer system, the data indicative of the analyte level of the patient with the external patient identifier associated with the at least one practice.

According to an aspect of some embodiments, the method further includes the step of associating, by the site server, a database identifier that is transferred to the database with the unique external identifier medical data set.

According to an aspect of some embodiments, the method further includes the step of storing, by the database, the external patient identifier medical data set according to the database identifier.

According to an aspect of some embodiments, the method further includes the step of associating, by the site server, a patient identifier with the external patient identifier or the external patient identifier analyte data set.

According to an aspect of some embodiments, the at least one practice is at least one hospital.

According to an aspect of many embodiments, a system for sharing data includes a sensor control device comprising an analyte sensor, wherein at least a portion of the analyte sensor is configured to be in fluid contact with a bodily fluid of a monitored user; a reader device configured to wirelessly receive data indicative of an analyte level of a patient from the sensor control device, wherein the reader device is further configured to send the data indicative of the analyte level; a first trusted computer system configured to: receive the data indicative of the analyte level; associate the data indicative of the analyte level with an external patient identifier associated with at least one practice to create an external patient identifier analyte data set; and transfer the external patient identifier analyte data set; a second trusted computer system configured to: receive the external patient identifier analyte data set; store the external patient identifier analyte data set in a folder associated with the at least one practice; and transfer the external patient identifier analyte data set that was stored in the folder associated with the at least one practice; a site server configured to: receive the external patient identifier analyte data set that was stored in the folder associated with the at least one practice; merge the external patient identifier analyte data set with an electronic medical record associated with the patient to create an external patient identifier medical data set; and transfer the external patient identifier medical data set to a database.

According to an aspect of many embodiments, a system for sharing data includes: a reader device configured to wirelessly receive data indicative of an analyte level of a patient from a sensor control device, wherein the reader device is further configured to send the data indicative of the analyte level; a first trusted computer system configured to: receive the data indicative of the analyte level; associate the data indicative of the analyte level with an external patient identifier associated with at least one practice to create an external patient identifier analyte data set; and transfer the external patient identifier analyte data set; a second trusted computer system configured to: receive the external patient identifier analyte data set; store the external patient identifier analyte data set in a folder associated with the at least one practice; and transfer the external patient identifier analyte data set that was stored in the folder associated with the at least one practice; a site server configured to: receive the external patient identifier analyte data set that was stored in the folder associated with the at least one practice; merge the external patient identifier analyte data set with an electronic medical record associated with the patient to create an external patient identifier medical data set; and transfer the external patient identifier medical data set to a database.

According to an aspect of some embodiments, the sensor control device comprising an analyte sensor, wherein at least a portion of the analyte sensor is configured to be in fluid contact with a bodily fluid of a monitored user;

According to an aspect of many embodiments, a method for enrolling a patient in a data sharing program includes the steps of: accepting a first invitation to join a practice in an analyte monitoring program; sending a second invitation to a patient in the analyte monitoring program, wherein the second invitation requests consent to share data with the practice; and in response to the patient accepting the second invitation, enrolling the patient in a program, wherein enrolling in the program enables analyte data of the patient to be shared to a third party database, and wherein the patient is associated with an external patient identifier in the analyte monitoring program.

According to an aspect of some embodiments, the patient is enrolled when an external patient identifier associated with the patient is entered to confirm enrollment.

According to an aspect of some embodiments, the second invitation is displayed in a modal in the analyte monitoring program to the patient.

According to an aspect of some embodiments, the second invitation is displayed in an email sent from the analyte monitoring program to the patient.

Many of the embodiments provided herein are GUI features for providing consent or address security issues that are highly intuitive, user-friendly, and provide for the sharing and rapid access to physiological information of a user. More specifically, these embodiments allow a user to easily navigate through and between different user interfaces that can quickly allow users and administrators to manage invitations and access to medical data, including analyte levels, glucometrics, and electronic medical records.

The improvements to the aforementioned features and GUIs in the various aspects described and claimed herein produce a technical effect at least in that they assist the user of the device to operate the device more accurately, more efficiently, and more safely. It will be appreciated that the information that is provided to the user via the GUI, the order in which that information is provided, and the clarity with which that information is structured can have a significant effect on the way the user interacts with the system and the way the system operates. The report GUI therefore guides the user in the technical task of operating the system to obtain the necessary permissions and/or obtain information accurately and efficiently. Other improvements and advantages are provided as well. The various configurations of these devices are described in detail by way of the embodiments which are only examples.

Other systems, devices, methods, features and advantages of the subject matter described herein will be or will become apparent to one with skill in the art upon examination of the following figures and detailed description. It is intended that all such additional systems, devices, methods, features, and advantages be included within this description, be within the scope of the subject matter described herein, and be protected by the accompanying claims. Aspects of the embodiments are set out in the independent claims and preferred features are set out in the dependent claims. The preferred features of the dependent claims may be provided in combination in a single embodiment and preferred features of one aspect may be provided in conjunction with other aspects. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of an analyte monitoring system comprising a sensor applicator, a sensor control device, a reader device, a network, a trusted computer system, and a local computer system.

FIG. 2A is a block diagram depicting an example embodiment of a reader device.

FIGS. 2B and 2C are block diagrams depicting example embodiments of sensor control devices.

FIGS. 3A and 3B are overviews of exemplary systems that include an analyte monitoring data management and electronic medical records management.

FIG. 3C is an exemplary method for sharing analyte data and medical records.

FIGS. 4A and 4B are exemplary methods for linking the accounts in an analyte monitoring program and a database.

FIGS. 5A-5E are example embodiments of various graphical user interfaces (“GUIs”) and reporting GUIs, related to linking a patient's analyte monitoring account with a database.

FIGS. 6A-6F are example embodiments of various GUIs related to linking a patient's analyte monitoring account with a database in a mobile application.

FIG. 7 is an example method for utilizing a two-factor authentication scheme with an analyte monitoring system.

FIGS. 8A and 8B are example embodiment of various GUIs relating to 2FA schemes used in an analyte monitoring system.

FIG. 9 is an example embodiment of an account settings GUI for a 2FA scheme to be used in an analyte monitoring system.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include GUIs, software, and digital interfaces for analyte monitoring systems, and methods and devices relating thereto. Accordingly, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices, reader devices, local computer systems, and trusted computer systems are disclosed, and these devices and systems can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps.

Improved reporting graphical user interfaces for analyte monitoring systems are provided. For example, disclosed herein are various embodiments of graphical user interfaces (“GUIs”). The GUIs are highly intuitive, user-friendly, and provide for rapid access to physiological information of a user. In sum, these embodiments provide for a robust, user-friendly interfaces that can increase user engagement with the analyte monitoring system and provide for timely and actionable responses by the user, to name a few advantages. Other improvements and advantages are provided as well. The various configurations of these devices are described in detail by way of the embodiments which are only examples.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

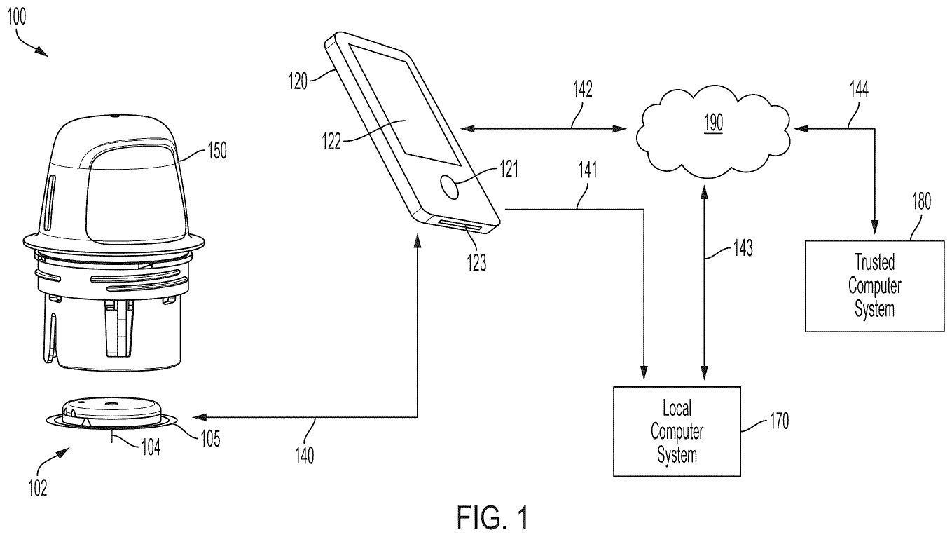

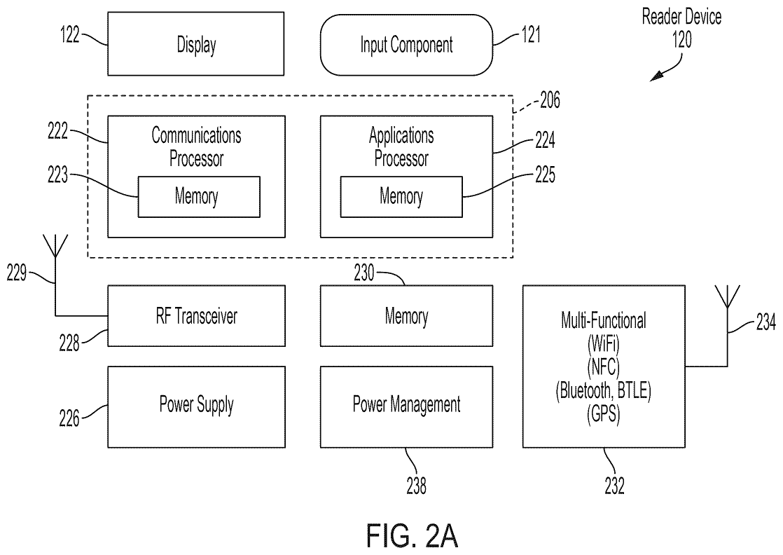

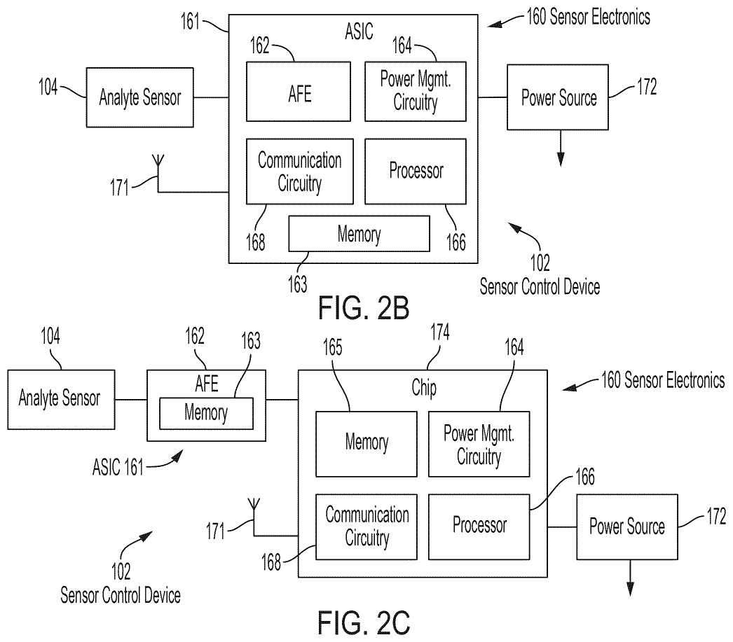

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can view and use applications installed in memory on reader device 120 using screen 122 (which, in many embodiments, can comprise a touchscreen), and input 121. A device battery of reader device 120 can be recharged using power port 123. While only one reader device 120 is shown, sensor control device 102 can communicate with multiple reader devices 120. Each of the reader devices 120 can communicate and share data with one another. More details about reader device 120 is set forth with respect to FIG. 2A below. Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth. A trusted computer system 180 can include a cloud-based platform or server, and can provide for authentication services, secured data storage, report generation, and can communicate via communications path 144 with network 190 by wired or wireless technique. In addition, although FIG. 1 depicts trusted computer system 180 and local computer system 170 communicating with a single sensor control device 102 and a single reader device 120, it will be appreciated by those of skill in the art that local computer system 170 and/or trusted computer system 180 are each capable of being in wired or wireless communication with a plurality of reader devices and sensor control devices.

### Example Embodiment of Reader Device

FIG. 2A is a block diagram depicting an example embodiment of a reader device 120, which, in some embodiments, can comprise a smart phone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further, reader device 120 can also include a multi-functional transceiver 232, which can comprise wireless communication circuitry, and which can be configured to communicate over Wi-Fi, NFC, Bluetooth, BTLE, and GPS with an antenna 234. As understood by one of skill in the art, these components are electrically and communicatively coupled in a manner to make a functional device.

### Example Embodiments of Sensor Control Devices

FIGS. 2B and 2C are block diagrams depicting example embodiments of sensor control devices 102 having analyte sensors 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data. According to some embodiments, for example, a current glucose value can be transmitted from sensor control device 102 to reader device 120 every minute, and historical glucose values can be transmitted from sensor control device 102 to reader device 120 every five minutes.

In some embodiments, to conserve power and processing resources on sensor control device 102, digital data received from AFE 162 can be sent to reader device 120 (not shown) with minimal or no processing. In still other embodiments, processor 166 can be configured to generate certain predetermined data types (e.g., current glucose value, historical glucose values) either for storage in memory 163 or transmission to reader device 120 (not shown), and to ascertain certain alarm conditions (e.g., sensor fault conditions), while other processing and alarm functions (e.g., high/low glucose threshold alarms) can be performed on reader device 120. Those of skill in the art will understand that the methods, functions, and interfaces described herein can be performed—in whole or in part—by processing circuitry on sensor control device 102, reader device 120, local computer system 170, or trusted computer system 180.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 may include memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiments of Systems for Integrating Analyte Monitoring Data and Electronic Medical Record Data

Described herein are example embodiments of systems for integrating and sharing analyte monitoring data and electronic medical records data.

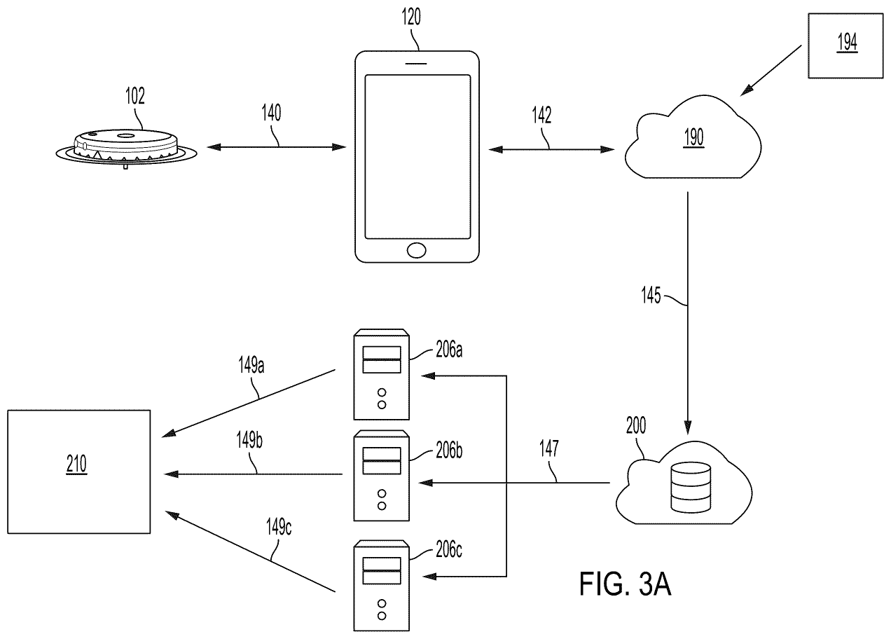

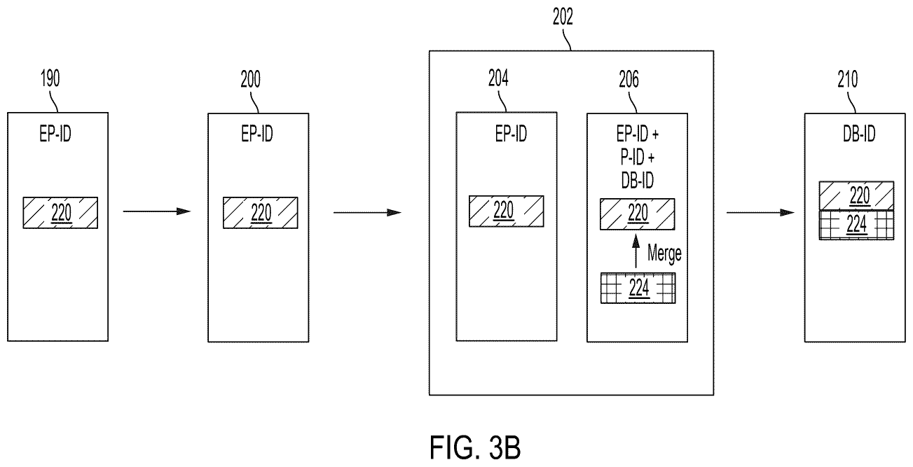

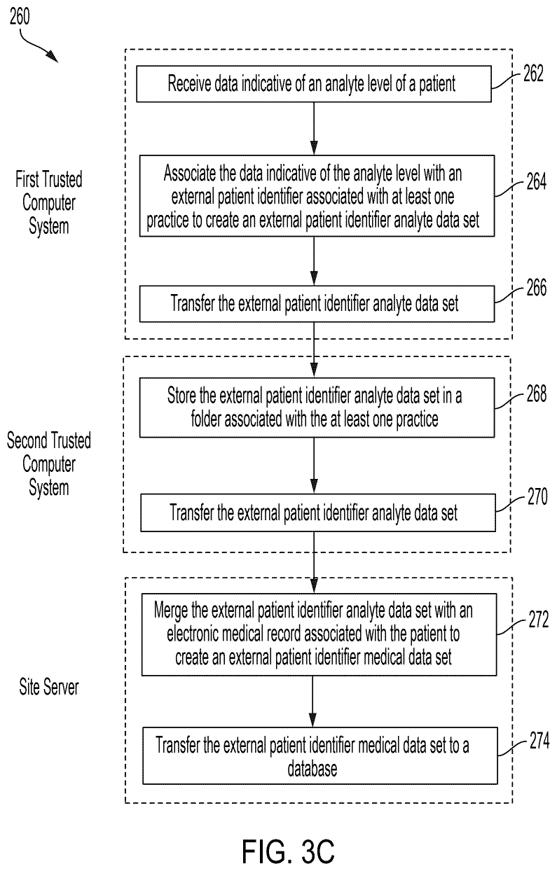

FIG. 3A is a conceptual diagram depicting an example embodiment of a data flow diagram that merges data related to analyte levels of a patient with the patient's corresponding electronic medical records. As described elsewhere in the application, sensor control device 102 can be applied to a patient's skin where a sensor 104 is maintained in position for a period of time by an adhesive patch. Sensor control device 102 can communicate with reader device 120 via a communication path 140 using a wired or wireless technique. Reader device 120 can communicate via a communications path 142 with a first trusted computer system 190, by a wired or wireless communication protocol as described previously. First trusted computer system 190 can include a cloud-based platform or server, and can provide for authentication services, secured data storage, and report generation. First trusted computer system 190 can communicate via communications path 145 with second trusted computer system 200 by wired or wireless technique. In some embodiments, second trusted computer system 200 can be located on a separate network (e.g., separated by one or more firewalls), or in a separate physical location from first trusted computer system 190. Second trusted computer system 200 can communicate via communication paths 147 to at least one site server 206a-206c by a wired or wireless communication protocol as described previously. In some embodiments, one or more site servers 206a-206c can be located on a separate network (e.g., separated by one or more firewalls), or in a separate geographical location from the second trusted computer system 200. In some embodiments, first and second trusted computer systems 190, 200 may be cloud servers. The at least one site server 206a-206c can communicate via communication paths 149a-149c to a database 210.

A patient may have an account associated with an analyte monitoring application on the reader device 120 that receives the data related to analyte levels from the sensor control device 102. If the patient is under the care or supervision of a healthcare center, hospital, or practice, the patient may receive an invitation 194 to share their data with the practice (or multiple practices). If the patient accepts the invitation 194 and consents to share their data related to analyte levels, the first trusted computer system 190, which receives the data from the reader device 120, may transmit at least a portion of the patient's analyte monitoring data to a second trusted computer system 200. The second trusted computer system 200 may have an individual and separate store (e.g., folder) for each practices, and the second trusted computer system 200 may store the portion of the patient's analyte monitoring data in the folder corresponding to the practice associated with the invitation. According to another aspect of the embodiments, the analyte monitoring data associated with the patient may be labeled with an external patient identifier unique to that particular patient. The second trusted computer system 200 may then transmit data stored in each of the separate folders to a site server 206a-206c of the corresponding practice. In some embodiments, the second trusted computer system 200 does not transmit patient data back to the first trusted computer system 190. The second trusted computer system 200 may be designed such that data from different practices are not co-mingled. The site server 206a-206c may merge the data related to analyte levels with electronic medical records for the patient, or the analyte data may be shared with the hospital practice's electronic medical records system. The merged data (analyte and EMR data) may then be transferred to a database 210.

As seen in FIG. 3B, various patient identifiers may be assigned to the data and used in lieu of personal identification. The first trusted computer system 190 may associate an external patient identifier (“EP-ID”), a pseudonymized patient ID, with a patient's analyte data 220 received from the analyte monitoring system. The EP-ID may be assigned to the patient by a practice (e.g., hospital) after the patient accepts an invitation from the practice to share their analyte data. Alternatively, the patient may already have an EP-ID, and this EP-ID may be entered into the analyte monitoring program. The first trusted computer system 190 may transfer the analyte data 220 associated with the patient's EP-ID to the second trusted computer system 200. In some embodiments, no personal identification is associated with the data 220 transferred to the second trusted computer system 200. The first trusted computer system 190 may transfer the data periodically, e.g., once a day in a nightly upload, to the second trusted computer system 200.

According to some embodiments, the second trusted computer system 200 may store the received data in the appropriate folder(s) associated with the EP-ID of the analyte data set 220. The analyte data set 220 may include one or more .CSV files containing the analyte (e.g., glucose) data. In some embodiments, each CSV file may include the EP-ID in the naming structure of the file. The data that is sent may include at least one of historical glucose results (e.g., glucose levels and time stamps from every 15 minutes), glucometrics, and reports. The glucometrics may include at least one of average glucose, time in range statistics, standard deviation statistics, and variability statistics. The reports may include AGP reports, daily pattern reports, and daily pattern reports. In some embodiments, no patient identity information is stored with the analyte data set 200. According to some embodiments, the files stored on the second trusted computer may be encrypted. In other embodiments, the communication link between the first trusted computer system 190 and second trusted computer system 200 may be encrypted, in addition to, or in lieu of, encrypting the filed stored on the second trusted computer system.

In some embodiments, the practice or hospital network 202 may have two servers 204, 206, wherein first server 204 is directly facing the Internet (e.g., in a DMZ network), and second server 206 does not directly interface with the Internet. Alternatively, in other embodiments, the practice or hospital 202 may only have a single server. The site server(s) 206 may manage the integration of the analyte monitoring system data 220 with the corresponding electronic medical records 224 of the patient associated with the EP-ID. The site server 206 may also associate or assign a patient identifier (“P-ID”), that is, a patient identifier associated with the electronics medical record system of the site server 206, with the analyte data set 220 and/or the corresponding EP-ID. The site server 206 may merge the electronic medical records 224 with the analyte data set 220. The site server 206 may also associate or assign a database identifier (“DB-ID”) with the analyte data set 220 and/or the corresponding EP-ID. In some embodiments, all or a portion of the practice or hospital network 202 is not online to ensure patient privacy.

The site servers 206 may transfer the merged data (including the analyte data set 220 and the corresponding electronic records 224) to a database 210. In some embodiments, database 210 may reside on a server located on a separate network (e.g., separated by one or more firewalls), or in a separate physical location from the practice or hospital network 202. The database 210 may include an entry server to receive the data, a multipurpose clinical data repository to store the data, and an analysis database to analyze the data.

In exemplary method 260, as depicted in FIG. 3C, in step 262, the first trusted computer system receives data indicative of an analyte level of a patient. In step 264, the first trusted computer system associates the data indicative of the analyte level of the patient with an external patient identifier associated with at least one practice to create an external patient identifier analyte data set. In step 266, the first trusted computer system transfers the external patient identifier analyte data set to a second trusted computer system. In step 268, the second trusted computer system stores the external patient identifier analyte data set in a folder associated with the at least one practice. In step 270, the second trusted computer system transfers the external patient identifier analyte data set to a site server associated with the at least on practice. In step 272, the site server merges the external patient identifier analyte data set with an electronic medical record associated with the patient to create an external patient identifier medical data set. In step 274, the site server transfers the external patient identifier medical data set to a database.

### Example Embodiments of Patient Enrollment and Consent Processes

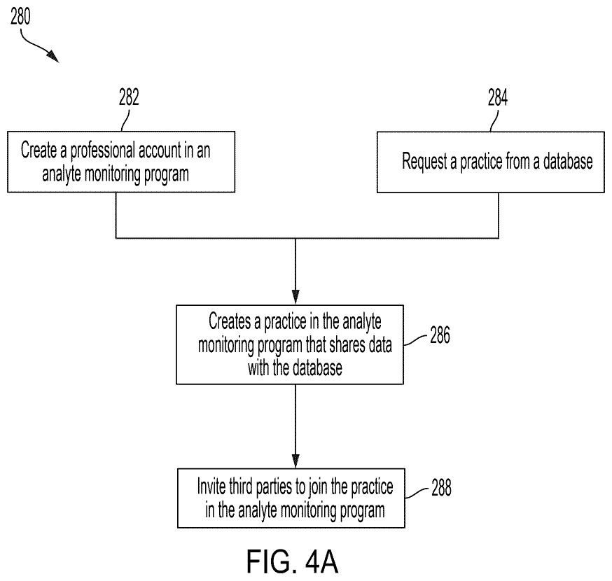

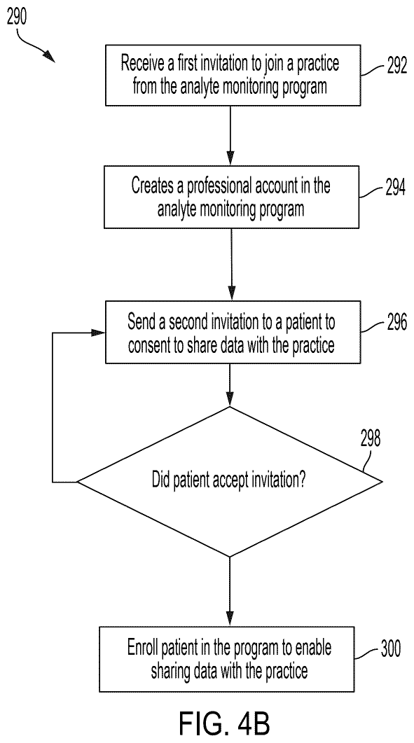

Hospitals may create a practice account that allows them to invite third parties, such as a healthcare research team, a care team, hospital administrators, and HCPs, who are then able to invite patients to share their data with a database. The data shared may include data related to their analyte levels and also electronic medical records. As seen in FIG. 4A, in step 282 of method 280, the hospital may create a professional account in an analyte monitoring program, which receives the data indicative of analyte levels from the sensor control device. In step 284, the hospital may also request a practice be registered with a database sharing program separate from the analyte monitoring program. In step 286, the hospital may create a practice in the analyte monitoring program that is configured to allow sharing of the data associated with the practice. In step 288, the hospital may invite third parties (such as healthcare research team, a care team, hospital administrators, and HCPs) to join the practice.

Once the HCP has received an invitation to join the practice, the HCP may invite their patients to enroll in the practice and allow their data to be shared with the healthcare practice, which may then share the data with the database 210. As seen in FIG. 4B, in step 292 of method 290, the HCP may receive an invitation to a practice from the analyte monitoring program. In one embodiment, if the HCP does not already have an account with the analyte monitoring program, the HCP may create an account (e.g., a professional account) in step 294. Step 294 may be skipped if the HCP already has an account in the analyte monitoring program. In step 296, the HCP may invite a patient(s) to the practice in the analyte monitoring program, which would allow the program to share the patient's data with the healthcare practice, which may then share the data with the database. If the patient accepts the invitation (step 296), then the HCP can enroll the patient in the practice (step 300). In one embodiment, the HCP may enter the patient's EP-ID to enroll the patient in the practice. In another embodiment, the HCP may have previously entered the EP-ID of the patient (or the EP-ID may already be associated with the patient's account in the analyte monitoring program), and the HCP must simply confirm that the patient should be enrolled (e.g., by clicking “enroll” at a prompt or by re-entering the patient's EP-ID). If the patient declines the invitation at step 298, then the HCP can re-send another invitation to the patient (in step 296) at a later time.

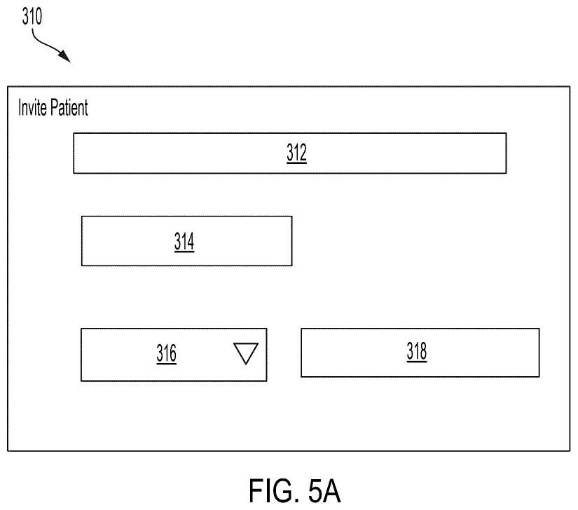

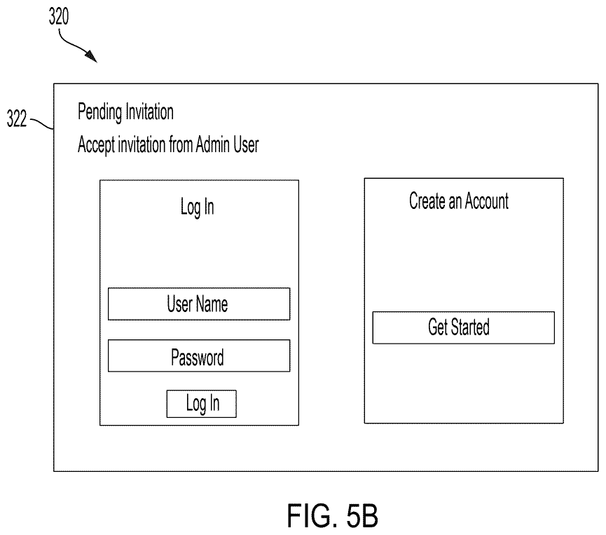

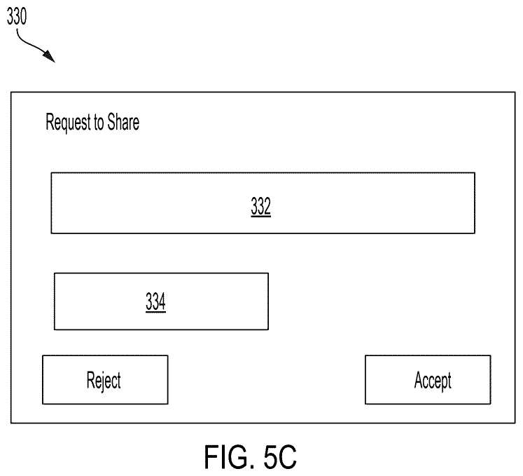

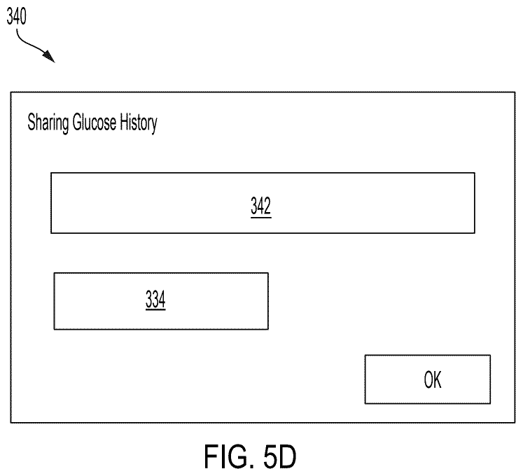

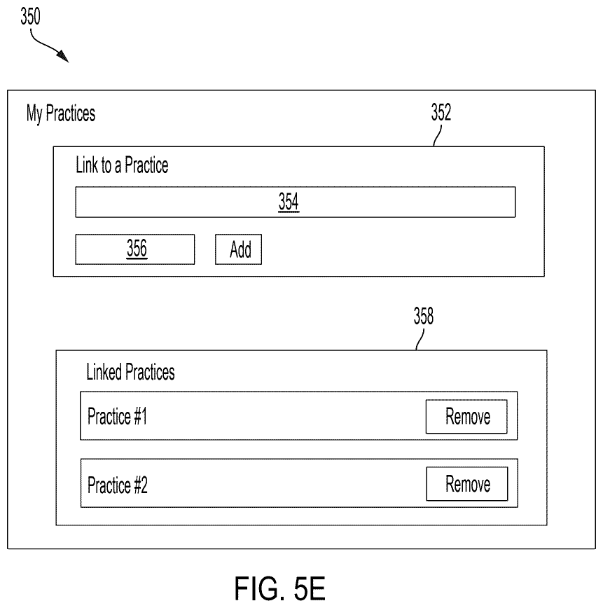

In the analyte monitoring program, the HCP may be presented with a modal or pop-up window that enables the HCP to enter requested information about the patient that is necessary for the invitation. As seen in FIG. 5A, the modal 310 may include field(s) 312 for entering the patients name, field 314 for entering the patient's date of birth, field 316 for entering the practice, and field 318 for entering the patient's email address associated with their analyte monitoring program account. The field 316 for entering the practice may be include a drop-down menu with a list of practices and may also, alternatively or in addition to, prepopulate the field as the HCP begins to type in a name of a practice. The invite/create patient modal 310 may be accessed through a create a new patient link from a link upload to patient screen or from an invite patient button on a global navigation bar in the analyte monitoring program.

After the invitation 194 is sent, the log in screen may indicate that there is an outstanding invitation. As seen in FIG. 5B, login screen for the analyte monitoring program may include a notification 322 that there is a “Pending Invitation” and may further suggest that the patient accept the invitation from the HCP, and may list the name of the requestor.

After the patient logs into the analyte monitoring program, as seen in FIG. 5C, a share request modal 330 may appear that indicates in text 332 that the third party (including the name of the requestor) is requesting access to the patient's glucose history for the following care teams, with the specific practice 334 to which the data will be shared listed below. If the patient accepts the request, then a confirmation modal 340 (seen in FIG. 5D) may appear that states in text 342 that the patient is sharing their glucose history with the practice 334 listed below. After the invitation is accepted, the HCP or requestor may receive a message or notification (e.g., modal or email) that the patient has consented and is ready to be enrolled in the database program, which will share glucose data and an EP-ID with the database. The requestor may complete the enrollment by clicking “enroll” or may also have to enter the patient's EP-ID or re-enter the patient's EP-ID to complete enrollment. After the patient is enrolled, the requestor may receive a message or notification (e.g., modal or email) that the patient has been enrolled in the program to share data with the database.

The patient can check to see which practices their account is linked to by accessing the My Practices tab under the Account Settings menu. The My Practices GUI 350 (see FIG. 5E) includes a section 352 that enables a patient to link their account to a practice without an invitation. The patient may enter the practice ID associated with the healthcare practice with which they want to share their data. The patient may get the practice ID from their HCP. After the patient enters the practice ID and clicks “Add”, the practice will be linked to their account. The My Practices GUI 350 also contains a second section listing linked practices 358. Each practice entry in the linked practices section 358 may include the practice name, address, phone number, and practice ID. A selectable button associated with each practice listing allows the user to remove the linked practice from their account, after which the patient's data would no longer be shared with the HCP practice or the database.

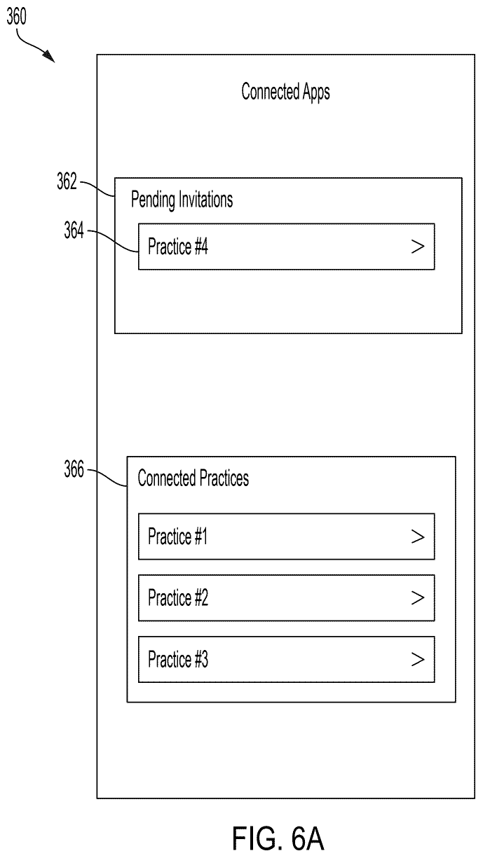

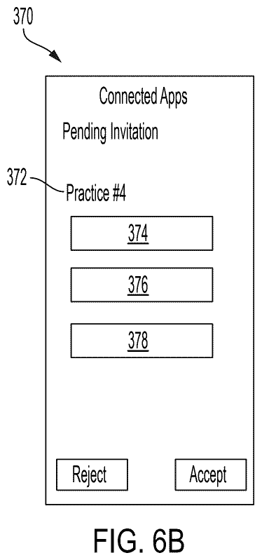

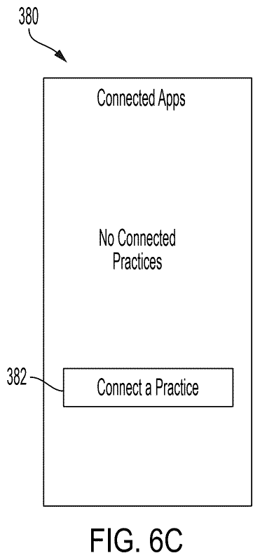

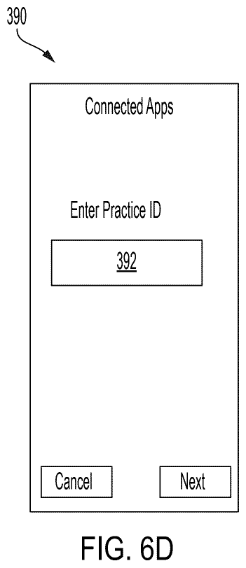

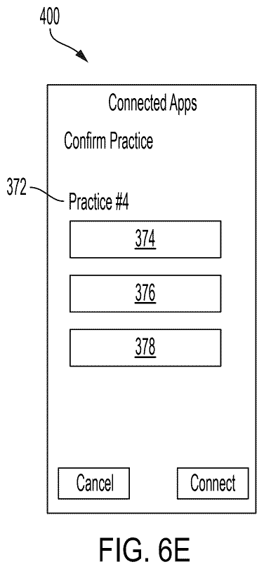

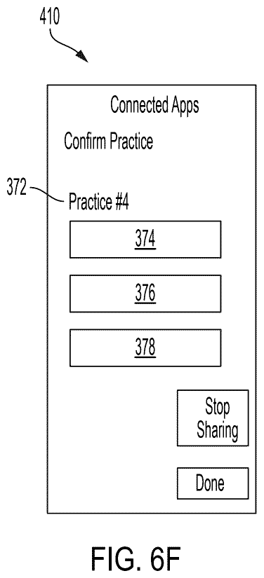

The patient may also link practices through the mobile application version of the analyte monitoring application. As seen in FIG. 6A, a Connected Applications GUI 360 may contain a pending invitations section 362 and a connected practices section 366. The pending invitations section 362 may list any practices that have sent an invitation to the patient to which the patient has not yet responded. The connected practices section 366 lists all of the practices that are currently linked to the patient's account. If the patient selects any of the practices listed in the pending invitations section 362 or linked practices section 366, a GUI displaying additional information regarding that practice will be displayed. If the patient selects a practice 364 listed in the pending invitations section 362, as seen in FIG. 6B, GUI 370 may appear that lists the name of the practice 372, and optionally may list the practice ID 374, address 376, and phone number 378. The patient may either accept or reject the invitation by selecting the appropriate button. Alternatively, if there are no pending invitations and no linked practices, the analyte monitoring application may display a Connected Apps GUI 380, as seen in FIG. 6C, that states that there are no connected practices and may further display a link 382 to connect a practice. If the user selects the link 382, a Connect Apps GUI 390 (see FIG. 6D) may appear that displays a field 392 in which the user may enter the practice ID number for the healthcare practice with which they want to share their data. After entering the practice ID, the analyte monitoring application may present Confirmation GUI 400 (see FIG. 6E) that lists the name of the practice 372, and optionally may list the practice ID 374, address 376, and phone number 378. If the patient wants to stop sharing their data with a practice that is currently linked to the account, the patient may select the practice from the connected practices section 366 of Connected Apps GUI 360 and GUI 410 may be displayed, as seen in FIG. 6F. Connected practice GUI 410 lists the name of the selected practice 372, and optionally lists the practice ID 374, address 376, and phone number 378. The user may select the option to stop sharing, and the practice will no longer be linked to the patient's account. Before the practice is unlinked, in some embodiments, a modal may appear asking for confirmation that the user wants to stop sharing their data with this practice and may only be unlinked after the patient confirms that they wish to stop sharing their data with the selected practice.

### Example Embodiments of Methods and Systems for Implementing Two-Factor Authentication

To enhance the security and integrity of analyte monitoring systems, such as those described above, it may be desirable to implement authentication schemes beyond simply requiring a username and password to access a user's analyte monitoring data. One such scheme involves the user of two-factor authentication (2FA), which requires that an individual trying to access a user's analyte monitoring data provide a username, password, and a second “factor” which is typically a verification code retrieved from a device associated with the individual (e.g., an e-mail account associated with the individual, an SMS text message sent to the individual's phone number, a code from an electronic key fob device, etc.). For some frequent users of analyte monitoring systems, however, it can be inconvenient and burdensome to login using 2FA each and every time they need access to a user's analyte monitoring data. For example, some HCPs may need to login multiple times a day to the same trusted computing system to access the analyte monitoring data of several patients. Therefore, there is a need for 2FA schemes in analyte monitoring systems that are robust and convenient for users.

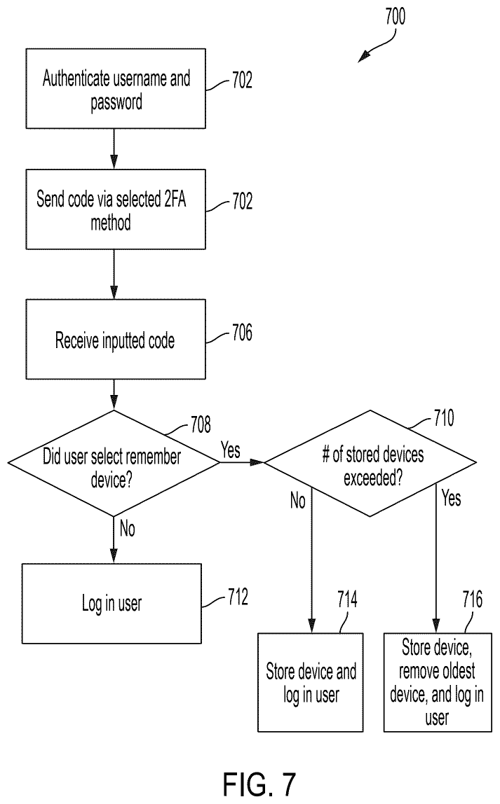

FIG. 7 is a flow diagram depicting an example method for a 2FA scheme for use with an analyte monitoring system. As an initial matter, those of skill in the art will appreciate that the method steps described herein can be instructions stored in memory of a reader device, trusted computer system, local computer system, or any computing device to be used with the analyte monitoring systems described herein. In some embodiments, the instructions, for example, can comprise an analyte monitoring software program. In some embodiments, the instructions can be graphical user interfaces (GUIs) to be generated on a trusted computer system that are then displayed or outputted to a user's reader device or local computing system.

Referring back to FIG. 7, at Step 702, a username and password is received and authenticated. In response thereto, at Step 704, a code is then transmitted to the user via a predetermined 2FA method. In some embodiments, the code can be sent to the user's e-mail address. In some embodiments, the code can be transmitted via SMS text to the user's telephone number. In still other embodiments, the code can be transmitted to an electronic key fob device associated with the user. At Step 706, the code is inputted by the user and received by the system.

At Step 708, a determination is made as to whether the user has selected an option to remember the device from which the user is logging in. If the user has not selected the “remember device” option, then the user is logged in at Step 712, and method 700 concludes. If the user has selected the “remember device” option, then, at Step 710, it is determined whether the user has already stored a maximum number of devices. If the user has not already stored the maximum number of devices, then, at Step 714, the system stores the current device in memory and logs the user in. If the user has already stored the maximum number of devices, then at Step 716, the oldest device is removed from memory, the current device is stored in memory, and the user is logged in.

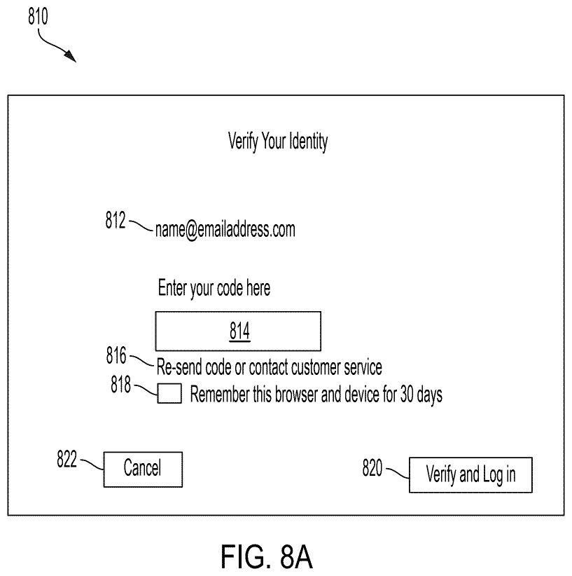

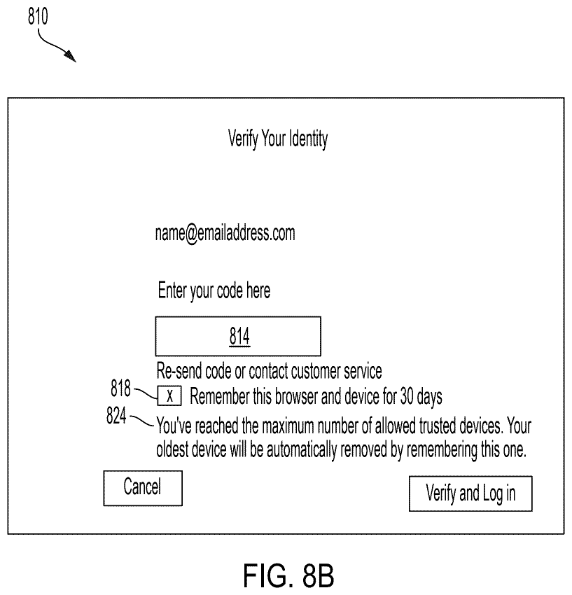

FIGS. 8A and 8B are example embodiments of graphical user interface 810 for use with a 2FA scheme, such as that described with respect to FIG. 7. Referring first to FIG. 8A, GUI 810 displays the 2FA method information 812 through which the user can expect to receive the code. According to some embodiments, GUI 810 also includes a field 814 to allow the user to enter the code when received. In some embodiments, GUI 810 can also include a link 816 (or button or other selectable object) to cause the system to re-send the code.

Referring still to FIGS. 8A and 8B, GUI 810 can also include a check box 818A (or button or other selectable object) to indicate that the user wishes to store the device in memory for thirty (30) days. Those of skill in the art will appreciate that other durations can be utilized (e.g., 7 days, 14 days, 21 days, 3 months). In some embodiments, the duration can be a fixed amount of time. In other embodiments, the duration can be a rolling window of time. For example, in some embodiments, the system may be configured to store a device in memory for fourteen (14) days on a rolling basis, such that the fourteen-day period restarts whenever the user logs in. In some embodiments, the duration can also be infinite, such that the device will be stored in memory until deleted or removed by the user or system. At the bottom of GUI 810, a cancel button 822 and login button 820 is provided.

According to some embodiments, and as described above with respect to FIG. 7, if the user selects box 818 (as shown in FIG. 8B), and a maximum number of devices have already been stored by the user, then a message 824 can be displayed indicating to the user that the current device will be stored and that the oldest device will automatically be removed.

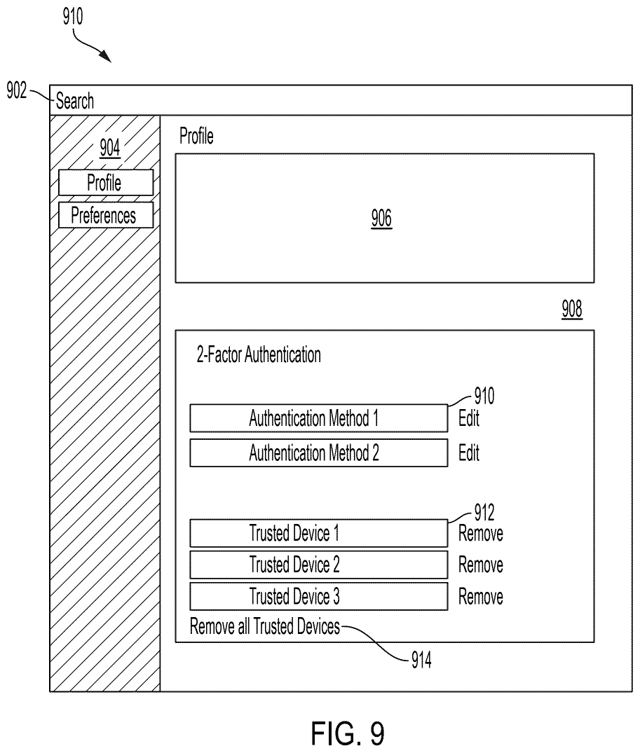

FIG. 9 is an example embodiment of an account settings GUI 910 configured for use with a 2FA scheme in an analyte monitoring system. In particular, account settings GUI 910 depicts an interface to allow a user to configure 2FA settings, among other account settings. According to some embodiments, for example, GUI 910 can include a search bar 902 and a navigation panel 904. In addition, in some embodiments, GUI 910 can include a profile section 906 that allows a user to change their password, change an e-mail address associated with the account, change other account information associated with the user, or to allow the user to delete the account.

According to another aspect of the embodiments, GUI 910 also comprises a 2FA section 908 to allow the user to configure settings relating to the 2FA scheme. In particular, 2FA section 908 includes an 2FA method portion 910 that allows a user to select one or more authentication methods by which the user may receive a 2FA code. Such methods may include, for example, via a telephone number or via an e-mail address. In some embodiments, 2FA method portion 910 can be configured to allow the user to edit the information associated with the 2FA method, or to indicate which 2FA method should be used as the primary and/or secondary method.

Referring still to FIG. 9, 2FA section 908 also includes a trusted device portion 912 that allows a user to review the number of trusted devices stored in memory. In some embodiments, trusted devices portion 912 also allows the user to remove individual devices. In some embodiments, trusted devices potion 912 can also include a link 914 (or button, or other selectable object) to allow the user to remove all trusted devices at once. According to some embodiments, a confirmation modal can be displayed if a user selects the link to remove all trusted devices.

It will be understood by those of skill in the art that any of the GUIs, reports interfaces, or portions thereof, as described herein, are meant to be illustrative only, and that the individual elements, or any combination of elements, depicted and/or described for a particular embodiment or figure are freely combinable with any elements, or any combination of elements, depicted and/or described with respect to any of the other embodiments.

It will also be understood by those of skill in the art that any of the GUIs, reports interfaces, or portions thereof, as described herein, can be implemented in an analyte monitoring system for monitoring one or more types of analytes. These analytes can include one or more of glucose, ketones, lactate, alcohol, or any other analytes that are detectable in a bodily fluid of the user. It will also be understood by those of skill in the art that the GUIs, reports interfaces, or portions thereof, as described herein, can incorporate data received from multiple analyte monitoring systems and devices relating thereto, including the incorporation of data from devices for taking ex vivo analyte measurements and/or physiological measurements.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

Systems and methods for integrating analyte monitoring system data and electronic medical record system data are described. The methods include associating a patient's analyte data identified with an external patient identifier associated with at least one healthcare practice. The patient's analyte data identified with the external patient identifier may be transferred from a first cloud server to a second cloud server. The analyte data may be stored in a folder associated with the at least one healthcare practice in the second cloud server. The analyte data may also be merged with electronic medical records of the patient in the second cloud server. The merged data may then be transferred to a database for further analysis.

### Clauses

Exemplary embodiments are set out in the following numbered clauses.t

1. A method of providing a database of medical data sets for sharing data comprising integrated analyte monitoring system data and electronic medical record system data, comprising the steps of:
receiving, by a first trusted computer system, data indicative of an analyte level of a patient from an analyte monitoring system;
determining an external patient identifier associated with at least one practice and associating, by the first trusted computer system, the data indicative of the analyte level of the patient with the external patient identifier associated with at least one practice to create an external patient identifier analyte data set;
transferring, from the first trusted computer system to a second trusted computer system, the external patient identifier analyte data set;
determining, by the second trusted computer system, a folder associated with the at least one practice, and storing, by the second trusted computer system, the external patient identifier analyte data set in the folder associated with the at least one practice;
transferring, from the second trusted computer system to a site server associated with the at least one practice, the external patient identifier analyte data set;
determining, by the site server, an electronic medical record associated with the patient, and merging, by the site server, the external patient identifier analyte data set with an electronic medical record associated with the patient to create an external patient identifier medical data set; and
transferring, by the site server, the external patient identifier medical data set to a database.
   2. The method of clause 1, wherein the external patient identifier analyte data set does not include identity information of the user.
   3. The method of clause 1 or 2, wherein at least a portion of the data related to the analyte levels of the user associated with the external patient identifier comprises historical glucose results.
   4. The method of clause 1, 2 or 3, wherein at least a portion of the data related to the analyte levels of the user associated with the external patient identifier comprises glucometrics.
   5. The method of clause 4, wherein the glucometrics comprises at least one of average glucose levels, time in range, and standard deviation.
   6. The method of any preceding clause, wherein at least a portion of the data related to the analyte levels of the user associated with the external patient identifier comprises an ambulatory glucose profile.
   7. The method of any preceding clause, wherein the external patient identifier is assigned by the at least one practice.
   8. The method of any preceding clause, wherein the data indicative of the analyte level is associated with a plurality of practices.
   9. The method of clause 8, wherein the second trusted computer system transfers the external patient identifier analyte data set to a site server associated with each of the plurality of practices.
   10. The method of any preceding clause, further comprising the step of inviting a user to share data with the at least one practice before the step of associating, by the first trusted computer system, the data indicative of the analyte level of the patient with the external patient identifier associated with the at least one practice.
   11. The method of any preceding clause, further comprising the step of associating, by the site server, a database identifier that is transferred to the database with the unique external identifier medical data set.
   12. The method of clause 11, further comprising the step of storing, by the database, the external patient identifier medical data set according to the database identifier.
   13. The method of any preceding clause, further comprising the step of associating, by the site server, a patient identifier with the external patient identifier or the external patient identifier analyte data set.
   14. The method of any preceding clause, wherein the at least one practice is at least one hospital.
   15. A system for providing a database of medical data sets for sharing data, comprising:
a sensor control device comprising an analyte sensor, wherein at least a portion of the analyte sensor is configured to be in fluid contact with a bodily fluid of a monitored user;
a reader device configured to wirelessly receive data indicative of an analyte level of a patient from the sensor control device, wherein the reader device is further configured to send the data indicative of the analyte level;
a first trusted computer system configured to:
receive the data indicative of the analyte level;
determine an external patient identifier associated with the at least one practice, and associate the data indicative of the analyte level with the external patient identifier associated with at least one practice to create an external patient identifier analyte data set; and
transfer the external patient identifier analyte data set;
a second trusted computer system configured to:
   receive the external patient identifier analyte data set;
   determine a folder associated with the at least one practice, and store the external patient identifier analyte data set in a folder associated with the at least one practice; and
   transfer the external patient identifier analyte data set that was stored in the folder associated with the at least one practice;
a site server associated with the at least one practice configured to:
   receive the external patient identifier analyte data set that was stored in the folder associated with the at least one practice;
   determine an electronic medical record associated with the patient, and merge the external patient identifier analyte data set with an electronic medical record associated with the patient to create an external patient identifier medical data set; and
   transfer the external patient identifier medical data set to a database.
      16. A method for providing a database by enrolling a patient in a data sharing program, comprising the steps of:
accepting a first invitation to join a practice in an analyte monitoring program;
determining a patient in the analyte monitoring program and sending a second invitation to the patient in the analyte monitoring program, wherein the second invitation requests consent to share data with the practice; and
in response to the patient accepting the second invitation, enrolling the patient in a program, wherein enrolling in the program enables analyte data of the patient to be shared to a third party database, and determining an external patient identifier wherein the patient is associated with the external patient identifier in the analyte monitoring program.
   17. The method of clause 16, wherein the patient is enrolled when an external patient identifier associated with the patient is entered to confirm enrollment.
   18. The method of clause 16 or 17, wherein the second invitation is displayed in a modal in the analyte monitoring program to the patient.
   19. The method of clause 16, 17 or 18, wherein the second invitation is displayed in an email sent from the analyte monitoring program to the patient.

What is claimed is:

1. A method of providing a database of medical data sets for sharing data, comprising integrated analyte monitoring system data and electronic medical record system data, the method comprising the steps of:
receiving, by a first trusted computer system, data indicative of an analyte level of a patient from an analyte monitoring system;
determining an external patient identifier associated with at least one practice and associating, by the first trusted computer system, the data indicative of the analyte level of the patient with the external patient identifier associated with the at least one practice to create an external patient identifier analyte data set;
transferring, from the first trusted computer system to a second trusted computer system, the external patient identifier analyte data set;
determining, by the second trusted computer system, a folder associated with the at least one practice, and storing, by the second trusted computer system, the external patient identifier analyte data set in the folder associated with the at least one practice;
transferring, from the second trusted computer system to a site server associated with the at least one practice, the external patient identifier analyte data set;
determining, by the site server, an electronic medical record associated with the patient, and merging, by the site server, the external patient identifier analyte data set with an electronic medical record associated with the patient to create an external patient identifier medical data set; and
transferring, by the site server, the external patient identifier medical data set to a database.

2. The method of claim 1, wherein the external patient identifier analyte data set does not include identity information of the user.

3. The method of claim 1, wherein at least a portion of the data related to the analyte levels of the user associated with the external patient identifier comprises historical glucose results.

4. The method of claim 1, wherein at least a portion of the data related to the analyte levels of the user associated with the external patient identifier comprises glucometrics.

5. The method of claim 4, wherein the glucometrics comprises at least one of average glucose levels, time in range, and standard deviation.

6. The method of claim 1, wherein at least a portion of the data related to the analyte levels of the user associated with the external patient identifier comprises an ambulatory glucose profile.

7. The method of claim 1, wherein the external patient identifier is assigned by the at least one practice.

8. The method of claim 1, wherein the data indicative of the analyte level is associated with a plurality of practices.

9. The method of claim 8, wherein the second trusted computer system transfers the external patient identifier analyte data set to a site server associated with each of the plurality of practices.

10. The method of claim 1, further comprising the step of inviting a user to share data with the at least one practice before the step of associating, by the first trusted computer system, the data indicative of the analyte level of the patient with the external patient identifier associated with the at least one practice.

11. The method of claim 1, further comprising the step of associating, by the site server, a database identifier that is transferred to the database with the external patient identifier medical data set.

12. The method of claim 11, further comprising the step of storing, by the database, the external patient identifier medical data set according to the database identifier.

13. The method of claim 1, further comprising the step of associating, by the site server, a patient identifier with the external patient identifier or the external patient identifier analyte data set.

14. The method of claim 1, wherein the at least one practice is at least one hospital.

15. A system for providing a database of medical data sets for sharing data, comprising:
a sensor control device comprising an analyte sensor, wherein at least a portion of the analyte sensor is configured to be in fluid contact with a bodily fluid of a monitored user;
a reader device configured to wirelessly receive data indicative of an analyte level of a patient from the sensor control device, wherein the reader device is further configured to send the data indicative of the analyte level;
a first trusted computer system configured to:
receive the data indicative of the analyte level;
determine an external patient identifier associated with the at least one practice, and associate the data indicative of the analyte level with the external patient identifier associated with at least one practice to create an external patient identifier analyte data set; and
transfer the external patient identifier analyte data set;
a second trusted computer system configured to:
receive the external patient identifier analyte data set;
determine a folder associated with the at least one practice, and store the external patient identifier analyte data set in the folder associated with the at least one practice; and
transfer the external patient identifier analyte data set that was stored in the folder associated with the at least one practice;
a site server associated with the at least one practice configured to:
receive the external patient identifier analyte data set that was stored in the folder associated with the at least one practice;
determine an electronic medical record associated with the patient, and merge the external patient identifier analyte data set with an electronic medical record associated with the patient to create an external patient identifier medical data set; and
transfer the external patient identifier medical data set to a database.

16. A method for providing a database by enrolling a patient in a data sharing program, comprising the steps of:
accepting a first invitation to join a practice in an analyte monitoring program;
determining a patient in the analyte monitoring program and sending a second invitation to the patient in the analyte monitoring program, wherein the second invitation requests consent to share data with the practice; and
in response to the patient accepting the second invitation, enrolling the patient in a program, wherein enrolling in the program enables analyte data of the patient to be shared to a third party database, and determining an external patient identifier, wherein the patient is associated with the external patient identifier in the analyte monitoring program.

17. The method of claim 16, wherein the patient is enrolled when an external patient identifier associated with the patient is entered to confirm enrollment.

18. The method of claim 16, wherein the second invitation is displayed in a modal in the analyte monitoring program to the patient.

19. The method of claim 16, wherein the second invitation is displayed in an email sent from the analyte monitoring program to the patient.
